(19) United States
(12) Patent Application Publication
(10) Pub. No.: US 2023/0384106 A1
(43) Pub. Date: Nov. 30, 2023

(54) MOTOR ASSIST ANALYTIC SYSTEM FOR AN ELECTRIC BICYCLE

(71) Applicant: Garmin International, Inc., Olathe, KS (US)

(72) Inventors: Sarah E. Fanning, Olathe, KS (US); Jordan K. Cowen, Salt Lake City, UT (US); Karen J. Moore, Prairie Village, KS (US)

(21) Appl. No.: 18/324,534

(22) Filed: May 26, 2023

Related U.S. Application Data
(60) Provisional application No. 63/365,518, filed on May 31, 2022.

(51) Int. Cl. G01C 21/34; B62M 6/40; B62J 50/22; B62J 45/20; B60L 58/12; A61B 5/00

(52) U.S. Cl. CPC G01C 21/3469; B62M 6/40; B62J 50/22; B62J 45/20; B60L 58/12; A61B 5/0002; B60L 2200/12

(57) ABSTRACT

A portable electronic device configured to be used by a user riding an electric bicycle (eBike). The portable electronic device is configured to connect in data communication with the eBike; receive an indication of a selected route over which the user intends to ride the eBike; determine, from map data and user data, an anticipated amount of motor assistance to be provided by the electric motor during the user's ride over the selected route; obtain an indication of a charge level of the battery of the eBike; determine, based on the anticipated amount of motor assistance and the charge level of the battery, whether the charge level of the battery of the eBike is sufficient to complete the user's ride over the selected route; and present an indication of whether the charge level of the battery of the eBike is sufficient to complete the user's ride over the selected route.

### RELATED APPLICATION

The present non-provisional application claims priority benefit to U.S. Provisional Patent Application Ser. No. 63/365,518, filed on May 31, 2022, and entitled “EBIKE COMPUTER.” The entirety of the above-identified provisional patent application is hereby incorporated by reference into the present non-provisional application.

### FIELD

Embodiments of the present invention are directed to systems for analyzing motor assistance provided to a user riding an electric bicycle. More particularly, embodiments of the present invention are directed to systems configured to be mounted to an electric bicycle being operated by a user and configured to, inter alia, determine an estimated range of an electric bicycle and/or determine an appropriate amount of motor assistance the electric bicycle should provide on behalf of the user.

### DESCRIPTION OF RELATED ART

Cycling has become a popular exercise and entertainment activity. One relatively new type of bicycle that has become popular is an electric bicycle, which includes an electric motor that can selectively provide assistance (“motor assistance”) to a user when riding the electric bicycle. For example, the electric bicycle may be configured to function in a manner similar to that of a standard bicycle, such that the user can actuate pedals of the electric bicycle to propel bicycle along a route. However, when encountering a difficult portion of the route, the user may require (or desire) assistance from the motor of the electric bicycle to assist in propelling the bicycle to, thereby, reduce the amount of physical effort required of the user. As such, the electric motor can be engaged to assist with propelling the electric bicycle.

### SUMMARY

Embodiments of the present invention comprise a portable electronic device configured to be used by a user riding an electric bicycle (eBike). The eBike comprises a battery and an electric motor, with the electric motor being configured to selectively provide motor assistance to the user in propelling the eBike. The portable electronic device comprises one or more memory elements configured to store map data and user data. The map data comprises distance information and terrain information for a plurality of routes. The user data comprises information indicative of a fitness level of the user. The portable electronic device additionally comprises a display screen configured to present information to the user. The portable electronic device is configured to perform a number of steps, including connecting in data communication with the eBike. An additional step includes receiving an indication of a selected route over which the user intends to ride the eBike. An additional step includes determining, from the map data and the user data, an anticipated amount of motor assistance to be provided by the electric motor during the user's ride over the selected route. An additional step includes obtaining an indication, via the data communication with the eBike, of a charge level of the battery of the eBike. An additional step includes determining, based on the anticipated amount of motor assistance and the charge level of the battery, whether the charge level of the battery of the eBike is sufficient to complete the user's ride over the selected route. A further step includes presenting, via the display screen, an indication of whether the charge level of the battery of the eBike is sufficient to complete the user's ride over the selected route.

Embodiments of the present invention further include a portable electronic device configured to be used by a user riding an electric bicycle (eBike), with the eBike comprising a battery and an electric motor. The electric motor is configured to selectively provide motor assistance to the user in propelling the eBike. The portable electronic device comprises one or more memory elements, with the memory elements being configured to store map data and user data. The map data comprises distance information and terrain information for a plurality of routes. The terrain information comprises information associated with elevation changes for the plurality of routes. The portable electronic device further comprises a display screen configured to present information to the user. The portable electronic device is configured to connect in data communication with the eBike. The portable electronic device is additionally configured to receive an indication of a selected route over which the user intends to ride the eBike. The portable electronic device is additionally configured to determine, from the map data and the user data, an anticipated amount of motor assistance to be provided by the electric motor during the user's ride over the selected route. The user data comprises past performance data related to the user previously riding the eBike over the selected route or to the user previously riding the eBike over one or more other routes with similar terrain as the selected route. The portable electronic device is additionally configured to obtain an indication, via the data communication with the eBike, of a charge level of the battery of the eBike. The portable electronic device is additionally configured to determine, based on the anticipated amount of motor assistance and the charge level of the battery, whether the charge level of the battery of the eBike is sufficient to complete the user's ride over the selected route. The portable electronic device is further configured to present, via the display screen, an indication of whether the charge level of the battery of the eBike is sufficient to complete the user's ride over the selected route.

This summary is not intended to identify essential features of the present invention, and is not intended to be used to limit the scope of the claims. These and other aspects of the present invention are described below in greater detail.

### BRIEF DESCRIPTION OF THE DRAWINGS

Embodiments of the present invention are described in detail below with reference to the attached drawing figures, wherein:

FIG. 1 is a schematic diagram of components of an electric bicycle (eBike);

FIG. 2 is a perspective view of a portion of the eBike from FIG. 1, with a portable electronic device attached to the eBike via a connector;

FIG. 3 is a schematic diagram of components of the portable electronic device from FIG. 2;

FIG. 4 is a graphical user interface (GUI) displayable on a graphic display of the portable electronic device from FIGS. 2 and 3, with the GUI illustrating a graphical map with a selected route and with segments of the selected route being highlighted with a route-charge highlighting to illustrate an estimated charge level of a battery of the eBike from FIGS. 1 and 2 over the selected route;

FIG. 5 is a GUI illustrating an update to the graphical map and the route-charge highlighting from FIG. 4, after the user has begun riding the eBike over the selected route;

FIG. 6 is a GUI displayable on a graphic display of the portable electronic device from FIGS. 2 and 3, with the GUI illustrating a charge level of a battery of the eBike from FIGS. 1 and 2, an amount of motor assistance being provided by the eBike, and various ride data associated with the user's ride over a selected route; and

FIG. 7 is a GUI displayable on a graphic display of the portable electronic device from FIGS. 1 and 2, with the GUI illustrating ride data associated with the user's ride over a selected route having been completed.

The figures are not intended to limit the present invention to the specific embodiments they depict. While the drawings do not necessarily provide exact dimensions or tolerances for the illustrated structures or components, the drawings are to scale with respect to the relationships between the components of the structures illustrated in the drawings.

### DETAILED DESCRIPTION

The following detailed description of embodiments of the invention references the accompanying figures. The embodiments are intended to describe aspects of the invention in sufficient detail to enable those with ordinary skill in the art to practice the invention. The embodiments of the invention are illustrated by way of example and not by way of limitation. Other embodiments may be utilized and changes may be made without departing from the scope of the claims. The following description is, therefore, not limiting. The scope of the present invention is defined only by the appended claims, along with the full scope of equivalents to which such claims are entitled.

In this description, references to “one embodiment,” “an embodiment,” or “embodiments” mean that the feature or features referred to are included in at least one embodiment of the invention. Separate references to “one embodiment,” “an embodiment,” or “embodiments” in this description do not necessarily refer to the same embodiment and are not mutually exclusive unless so stated. Specifically, a feature, component, action, step, etc. described in one embodiment may also be included in other embodiments, but is not necessarily included. Thus, particular implementations of the present invention can include a variety of combinations and/or integrations of the embodiments described herein.

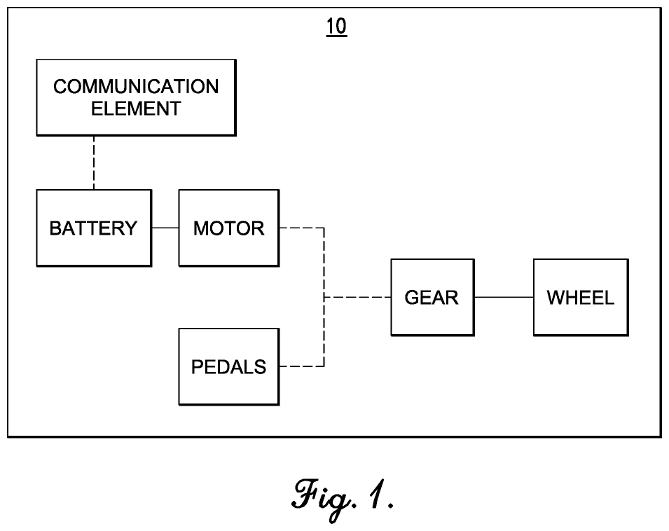

Embodiments of the present invention are directed to a motor assist analytic system for an electric bicycle (herein referred to as an “eBike”). An exemplary eBike 10 is schematically illustrated in FIG. 1 and may comprise common components that are generally associated with a standard bicycle, such as pedals operably engaged with one more wheels of the eBike 10 (perhaps through a gear system). As such, a user can acuate the pedals with the user's feet to cause the wheels to rotate and propel the eBike 10. In addition, however, the eBike 10 may also comprise an electric motor operably engaged with the wheels of the eBike 10 (perhaps through the gear system). The eBike 10 may further include one or more electric storage elements, such as batteries, that store electrical energy and provide such energy to the electric motor to power the motor. As such, the electric motor can selectively provide motor assistance to the user of the eBike 10 to selectively power the wheels of the eBike (perhaps through the gear system) to aid in propelling the eBike. The electric motor may be used to partially propel the eBike 10 (with the user actuating the pedals to provide any remaining power necessary to propel the eBike 10), or the electric motor may be used to fully propel the eBike 10.

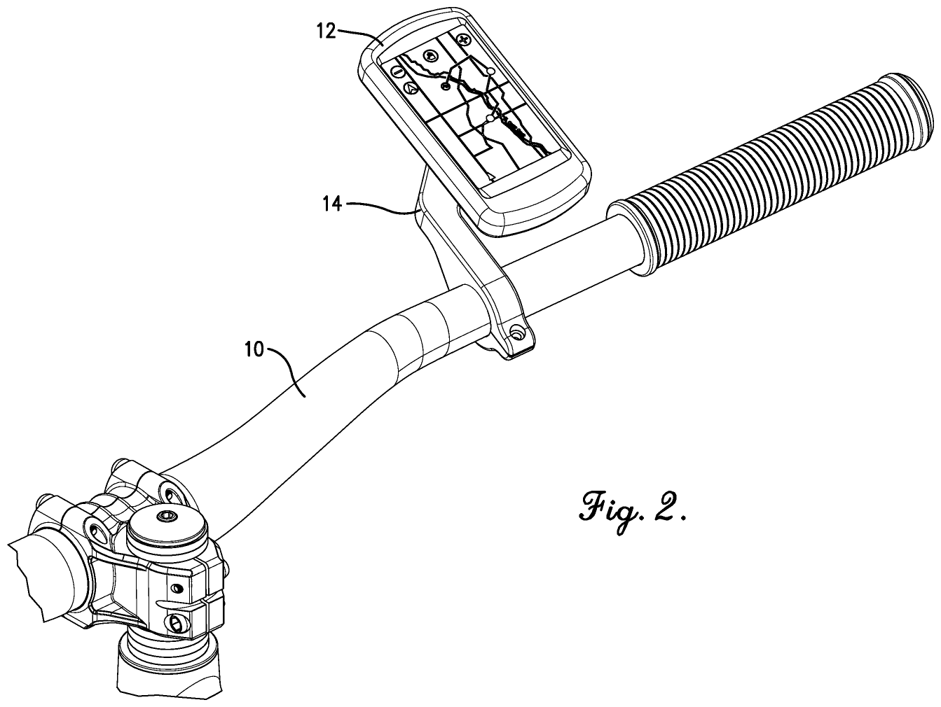

The motor assist analytic system of embodiments of the present invention may comprise a portable electronic device 12, as illustrated in exemplary fashion in FIG. 2, which can be used by the user riding the eBike 10 to determine an operating range of the eBike 10. As such, the device 12 can allow the user to plan and navigate routes with the eBike 10 providing the user with desired amounts of motor assistance from the eBike's 10 electric motor. The device 12 may, among other features described in more detail below, also be configured to track the user's previous usage of the eBike 10 and track various fitness metrics of the user, so as to estimate the amount of motor assistance the user is likely to demand as the user operates the eBike 10 over various routes. The portable electronic device 12 may comprise generally any type of portable electronic device, such as an eBike computer, a tablet, a smartphone, and/or a wearable device (e.g., a smartwatch). In some specific embodiments, such as illustrated in FIG. 2, the device 12 is configured to be removably coupled with the eBike 10. Such coupling may be facilitated by a connector 14 that interconnects the device 12 with the eBike 10 (e.g., via handlebars of the eBike 10).

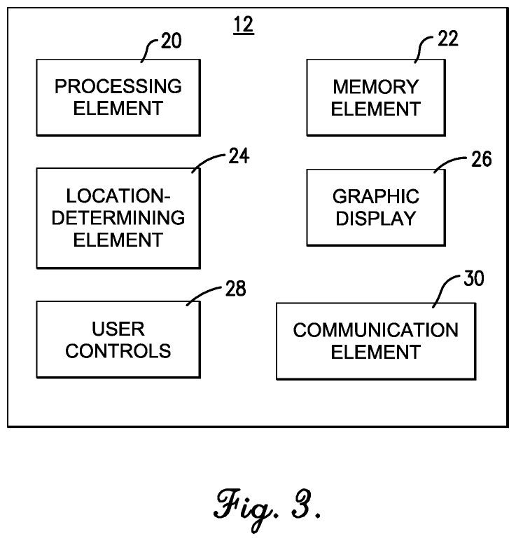

Turning to the device 12 in more detail, as schematically illustrated in FIG. 3, the device 12 may generally comprise a computing device that includes one or more processing elements 20, one or more memory elements 22, one or more location-determining elements 24, a graphic display 26, one or more user controls 28, and/or a communication element 30. The processing element 20 may implement operating systems, and may be capable of executing a computer program (which may be stored on the memory element 22), which is also generally known as instructions, commands, software code, executables, applications (“apps”), and the like. The processing element 20 may include processors, microprocessors, microcontrollers, field programmable gate arrays, and the like, or combinations thereof. The memory element 22 may be capable of storing or retaining the computer program and may also store data, typically binary data, including text, databases, graphics, audio, video, combinations thereof, and the like. The memory element 22 may also be known as a non-transitory “computer-readable storage medium” and may include random access memory (RAM), read only memory (ROM), flash drive memory, floppy disks, hard disk drives, optical storage media, and the like, or combinations thereof. In general, the memory element 22 may be configured to store all of the information described herein, which is used to carry out the methods and processes of embodiments of the present invention. Such methods and processes of the present invention, as described herein, may be performed (at least partly) by the processing element 20 of the device 12 executing the computer program stored on the memory element 22.

In addition to the above-described data, the memory element 22 may be configured to store map data, user data, bike data, and/or weather data. The map data may include cartography data and/or terrain data. Cartography data means data representative of one or more of the following features within a geographical area, which can be used by the processor 20 to generate a graphical map via the graphic display 26 (also referred to herein as “display screen”): topography, transportation lines (e.g., paved roads, non-paved roads (e.g., gravel roads, trails, paths, and the like, railroad lines, rivers, streams, etc.), political boundaries, place names, bodies of water, etc. The cartography data may also include location information for each of such features. Such location information may comprise location coordinates, such as latitude and longitude or the like. As a result, the device 12 may be configured to present the cartography data graphically on the graphic display 26, such as in the form of a graphical map. Furthermore, the device 12 may be configured to store and/or calculate distance information between various locations on the map and/or along transportation lines (or portions thereof). Thus, as used herein, distance information may comprise location information from which the device 12 may calculate distances. More particularly, the memory elements 22 may store distance information for a plurality of routes that extend along transportation lines.

As used herein, terrain data means data representative of various physical features associated with a transportation line (e.g., a street, a road, a path, or a trail) and/or with a section of a transportation line. In some embodiments, the terrain data may comprise one or more of the following for each transportation line (or portion thereof): length of the transportation line, grade of the transportation line, category rating of the transportation line and/or of the grade, elevation change of the transportation line, popularity of the transportation line, specific grade at a given location of the transportation line, and specific elevation at a given location of the transportation line. In some embodiments, some or all of the terrain data may be categorized as cartography data or may be derived from the cartography data. Furthermore, in some embodiments a section of a transportation may be defined as a “climb,” which refers to a portion of a transportation line in which altitude is gained. In certain embodiments, a portion of a transportation line may only be defined as a climb if the portion has a minimum length (e.g., at least 500 meters) and such portion exceeds a minimum average gradient (e.g., at least a 3% grade). Terrain data specifically associated with a climb may be referred to as climb data.

As will be described in more detail below, the user data may comprise various information about the user and/or the user's operation of the eBike that may be used by the device 12 to determine an anticipated amount of motor assistance. For instance, the user data may comprise information, such as fitness metrics, indicative of a fitness level of the user. In addition, the user data may comprise ride data related to the user's past rides on the eBike (e.g., past performance information). As also described in more detail below, bike data is information related to the eBike 10, while weather data is information related to the weather around the immediate area of the user and/or the eBike 10.

Returning to the components of the portable electronic device 12, the location-determining element 24 may comprise a Global Positioning System (GPS) receiver configured to receive and/or process signals received from GPS satellites. As such, the location-determining element 24 is operable to determine a geographic position of the device 12 through processing of the received signal data. The signals from the GPS satellites may include various data suitable for use in position determination, such as timing signals, ranging signals, ephemerides, almanacs, and so forth. However, it should be apparent that a wide variety of other positioning systems may also be employed, such as other global navigation satellite systems (GNSS), terrestrial based systems (e.g., wireless-phone based systems that broadcast position data from cellular towers), wireless networks that transmit positioning signals, and so on. Furthermore, location-determining element 24 may alternatively, or additionally, include one or more sensors (e.g., gyros, odometers, accelerometers, and/or magnetometers), that aid in generating the position and/or direction of travel of the portable electronic device 12.

As noted above, and as will be described in more detail below, the device 12 is configured to graphically present the cartography data and/or the terrain data via the graphic display 26. As such, the graphic display 26 may comprise any device configured to present data in a graphical format, such as an LCD (Liquid Crystal Diode) display, a TFT (Thin Film Transistor) LCD display, an LEP (Light Emitting Polymer) or PLED (Polymer Light Emitting Diode) display, and/or the like, which are configured to display text and/or graphical information to a user, such as in the form of a graphical user interface (GUI). The graphic display 26 may be backlit via a backlight such that it may be viewed in the dark or other low-light environments. In some embodiments, the graphic display 26 may be configured as a touchscreen to receive input (e.g., data, commands, etc.) from a user. For example, a user may operate the portable electronic device 12 by touching the touchscreen and/or by performing gestures on the touchscreen. In some embodiments, the touchscreen may be a capacitive touchscreen, a resistive touchscreen, an infrared touchscreen, combinations thereof, and the like. In alternative embodiments, or in addition, the portable electronic device 12 may also include the one or more user controls 28, which may comprise input/output (I/O) components, such as a keypad, buttons, a wireless input device, a thumbwheel input device, a trackstick input device, and the like. The user controls 28 may also include one or more audio I/O devices, such as a microphone, speakers, and the like.

Furthermore, the portable electronic device 12 may also include a communication element 30 configured to permit the device 12 to send/receive data between different devices (e.g., components/peripherals) and/or over one or more communications networks. The communication element 30 may include various communication components and functionality including, but not limited to: one or more antennas; a transmitter, receiver, and/or transceiver; a wireless radio; data ports; software interfaces and drivers; networking interfaces; data processing components; and so forth. The networks over which the communication element 30 may communicate include various wired and wireless networks, such as a local area network, a wide area network, an intranet, the Internet; a satellite network; a cellular network; a mobile data network; and the like. Specific examples wireless networks include, but are not limited to: networks configured for communications according to: one or more standard of the Institute of Electrical and Electronics Engineers (IEEE), such as 802.11 or 802.16 (Wi-Max) standards; Wi-Fi standards promulgated by the Wi-Fi Alliance; Bluetooth standards promulgated by the Bluetooth Special Interest Group; and so on. Wired communications are also contemplated such as through universal serial bus (USB), Ethernet, serial connections, and so forth.

In view of the motor assist analytic system described above, the portable electronic device 12 is configured to be used by a user riding an electric bicycle, e.g., eBike 10, to determine whether a present charge level of the battery of the eBike 10 is sufficient to complete a user's ride over a selected route. Beneficially, such a determination may be made based on (i) an anticipated amount of motor assistance to be provided by the electric motor of the eBike 10, and (ii) the present charge level of the eBike's 10 battery. In more detail, a user can use the device 12 to identify a selected route over which the user wishes to ride the eBike 10. Such an identification of a route may be made by the user interacting with the device 12. Specifically, the device 12 may display, via the graphical display 26, a graphical user interface (“GUI”) that presents a graphical map of the geographical area around the present location of the user and/or of the eBike 10 (as perhaps identified by the location-determining element 24 of the device 12). Using the graphical map, the user may trace one or more transportation lines (e.g., roads, trails, paths, etc.), using the user controls 28 and/or the user's fingers (in the case of the graphical display 26 being a touchscreen), to identify a selected route. Alternatively, the user may enter various waypoints (e.g., a starting point, an ending point, and/or one or more intermediate points), and the device 12 may identify the selected route as the route that connects such waypoints. In further alternatives, the user may select one or more preselected, previously-identified, and/or previously-ridden routes to be the selected route. In still further alternatives, the device 12 may recommend one or more routes to be the selected route. The device 12 may then determine if the battery of the eBike 10 has sufficient charge to complete the user's ride over the selected route using an anticipated amount of motor assistance.

To determine the charge level of the battery, the device 12 may be connected in data communication with the eBike 10. Such data communication may be via wired or wireless connection. Specifically, the communication element 30 of the device 12 may connect with a corresponding communication element of the eBike 10, such that the eBike 10 can provide a present and/or real-time charge level of the battery of the eBike 10 (e.g., state-of-charge of the battery). Thus, the communication element of the eBike may be associated with the battery of the eBike, with the electric motor of the eBike, and/or with some additional control element (e.g., a control processor) of the eBike. Regardless, the state-of-charge of the battery may be determined, either by the eBike 10 or by the device 12, using standard techniques, such as the Voltage method, the current integration method (e.g., Coulomb counting), or the like, or combinations thereof.

Determination of whether the battery of the eBike 10 has a sufficient charge needed to complete the user's ride over the selected route may also be based on an anticipated amount of motor assistance that the eBike 10 is likely required to provide to the user during the user's ride over the selected route. Such an anticipated amount of motor assist may be based on map data and user data. As noted above, map data may include distance information and terrain data related to transportation lines (e.g., streets, roads, paths, trails, etc.). For instance, the map data may comprise distance information and terrain data for a plurality of routes, including for the selected route identified by the user. The device 12 may use the distance information and terrain data to determine whether the eBike's 10 battery's has sufficient charge to complete the selected route. For instance, the longer the total distance of a selected route, the more charge the battery of the eBike 10 will be required to have so that the eBike 10 can provide the necessary amount of motor assistance to the user over the longer distance and/or over the longer period of time that is required to traverse the longer distance. Similarly, a selected route with more severe elevation changes, as perhaps indicated by the terrain data, will generally necessitate the eBike to provide more motor assistance to aid in the user traversing such severe elevation changes. Thus, in addition to the total distance of the selected route, the anticipate amount of motor assistance may be based on map data and/or terrain data in the form of the number or severity of elevation changes over the selected route, the number or grades of climbs included in the selected route, the type of road surface of the selected route, etc.

Similarly, user data may be used to determine the anticipated amount of motor assistance needed to be provided to the user as the user rides the eBike 10 over the selected route. Such user data may include fitness metrics that are indicative of a fitness level of the user. Such fitness metrics may include, for instance, the user's age, sex, average heart rate, body mass index, VO₂ max, or the like, or combinations thereof. In some instances, the less physically fit the user, the more motor assistance the eBike 10 will be required to provide to the user as the user traverses the selected route. In addition to fitness metrics, the user data may include information related to the user's previous rides with the eBike 10 (i.e., past “ride data”). As will be expanded in more detail below, ride data may include amounts of motor assistance the user used during past rides, with such past rides perhaps including the user previously riding the eBike 10 over the selected route and/or the user previously riding the eBike over a different route having similar terrain as the selected route (e.g., similar total distance, similar elevation changes, similar number, categories, or grades of climbs, similar types of road surfaces, etc.). Such ride data may include information related to other users previously riding their own eBikes over the selected route and/or over other routes route having similar terrain (i.e., ride data of other users' past rides).

The ability of the eBike 10 to complete the selected route using a given amount of motor assistance may also be based on characteristics of the eBike 10, which may be in the form of bike data that includes: a type of battery, a size of the battery (e.g., max voltage, capacity, energy density), a battery discharge curve. It should be understood that such battery-related information may be used to determine the current charge level. of the battery. Regardless, the bike data may also include other more general information related to the eBike 10, such a type of bicycle (e.g., make, model, and/or year of manufacture), weight of the bicycle, type/size of wheels of the bicycle, type of gearing used on the bicycle, etc. In some embodiments, the bike data for the eBike 10 may be stored in the memory elements 22 of the device 12. Furthermore, the device 12 may store bike data for multiple eBikes. As such, the user may be required to identify the particular eBike 10 that the user is intending to ride on the selected route. In other embodiments, the device 12 may automatically determine the particular eBike 10 (and its associated bike data) when the device 12 connects in data communication with the eBike 10.

Furthermore, in some embodiments, weather data may be used to determine the anticipated amount of motor assistance required to complete the selected route. Such weather data may comprise wind speed and/or direction, precipitation type and amount, cloud coverage, air temperature/humidity, etc. Such weather data may be obtained by the device 12 from a remote server using the communication element 30.

Thus, based on the map data, the user data, the bike data, and/or the weather data, the device may determine an anticipated amount of motor assistance that will be required by the user to complete a selected route. Once the device 12 has determined the anticipated amount of motor assistance, the device 12 will compare such anticipated amount with the charge level of the battery of the eBike 10 (as obtained from the eBike) to determine whether the charge level of the eBike 10 is sufficient to complete the user's ride over the selected route. In addition, the device 12 may present, via the graphic display 26, an indication of whether the charge level of the eBike 10 is sufficient to complete the user's ride over the selected route. If the charge level is insufficient, the device 12 may generate an alert (e.g., a textual alert, a graphical alert, an audible alert, and/or a tactile alert) to provide an indication to the user that the charge level of the eBike's 10 battery is insufficient to complete the selected route. The device 12 may also provide an indication of the actual range of the eBike 10 considering the current charge level of the battery. As such, the user can select a different route over which to travel using the eBike 10, and the device 12 can perform the above-described steps again to determine whether the current charge level of the eBike 10 is sufficient to complete the user's ride over the newly selected, different route. For instance, the user may manually identify a new route by creating the new route based on waypoints or by selecting one or more preidentified routes stored in the device 12. In some alternative embodiments, the device 12 may recommend one or more potential new routes for selection that can be completed using the current charge level of the eBike's 10 battery, as recently determined.

In further alternatives, the user can manually adjust the amount of motor assistance to be used over the initially selected route from the originally anticipated amount of motor assistance determined by the device 12. For example, if the device 12 alerts the user that the charge level of the eBike's 10 battery is insufficient to complete the selected route using the anticipated amount of motor assistance, the user can manually enter a reduced amount of motor assistance to be provided during the user's ride across the selected route. Alternatively, the user can potentially increase the amount of motor assistance to a higher level than the anticipated amount. After manually adjusting the amount of motor assistance, the device 12 can perform the above-described steps again to determine whether the current charge level of the eBike 10 is sufficient to complete the user's ride over the selected route using the manually updated amount of motor assistance. Similarly, an alert may be generated and presented on the graphic display 26 to provide an indication to the user as to whether the current charge level of the eBike's 10 battery is sufficient.

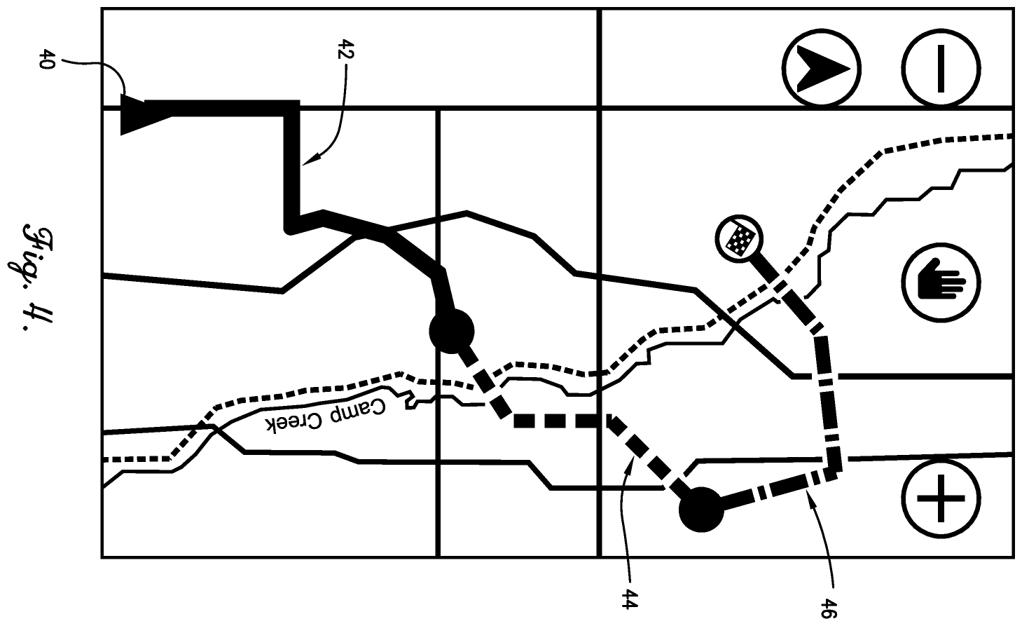

Upon the device 12 determining that the charge level of the eBike's 10 battery is sufficient to complete the selected route using the anticipated amount of motor assistance, an indication of such sufficiency may be provided to the user, e.g., via the graphic display 26. At such time, the device 12 may generate a GUI (displayed on the graphic display 26) with a graphic map of the geographic area immediately surrounding the user and/or the eBike 10 (as determined by the location-determining elements 24), with the selected route highlighted to indicate the direction the user should travel on the eBike 10 to follow the selected route (See, e.g., FIG. 4). In addition, the user's current position (as determined by the location-determining elements 24) may be shown in the displayed map via a user icon 40 (e.g., a triangular shaped icon).

In some embodiments, portions or segments of the selected route may be highlighted in different styles (e.g., line types, colors, gradients, etc.) to indicate an amount of charge the eBike's 10 battery is estimated to have as the user traverses along the selected route (referred to herein as “route-charge highlighting”). For example, FIG. 4 illustrates a GUI with an exemplary graphical map having the selected routes shown on the map. The selected route is segmented to illustrate a first half 42, a third quarter 44, and a fourth quarter 46 of the selected route. Along the first half 42 of the selected route, the eBike's 10 battery may be predicted by the device 12 to have a charge of more than fifty percent. As such, the first half 42 of the selected route may be highlighted with a solid line type (indicative of the battery having a sufficient state of charge). Alternatively, the first half 42 of the selected route may be highlighted with a particular color, such as the color green. Along the third quarter 44 of the selected route, the eBike's 10 battery may be predicted to have a charge of between twenty-five and fifty percent. As such, the third quarter 44 of the selected route may be highlighted with a dashed line type (indicative of the battery having a moderately deficient state of charge). Alternatively, the third quarter 44 of the selected route may be highlighted with a particular color, such as the color yellow. Finally, along the fourth, and final quarter 46 of the selected route, the eBike's 10 battery may be predicted to have a charge of between zero and twenty-five percent. As such, the fourth quarter 46 of the selected route may be highlighted with a dashed-dot-dash line type (indicative of the battery having a severely deficient state of charge). Alternatively, the fourth quarter 46 of the selected route may be highlighted with a particular color, such as the color red.

The above-described route-charge highlighting can be presented to the user so that the user can understand when the eBike's 10 battery could potentially run out of charge along the user's ride of the selected route. Thus, the route-charge highlighting functions as a battery confidence level. From the route-charge highlighting shown in FIG. 4, the user could infer that the eBike's 10 battery could potentially run out of charge during the fourth quarter 46 of the selected route. Such information may be beneficial for the user to plan the user's ride over the selected route. It should be understood that the above-described route-charge highlighting methodology is exemplary and is generated based on the eBike's 10 current battery charge, on the anticipated amount of motor assist, and on map data (e.g., length and terrain of the selected route). As a result, each individual route that may be selected by a user may have a unique route-charge highlighting. Furthermore, the same route may have different route-charge highlighting depending on the current charge level of the eBike's 10 battery and/or on the particular eBike used.

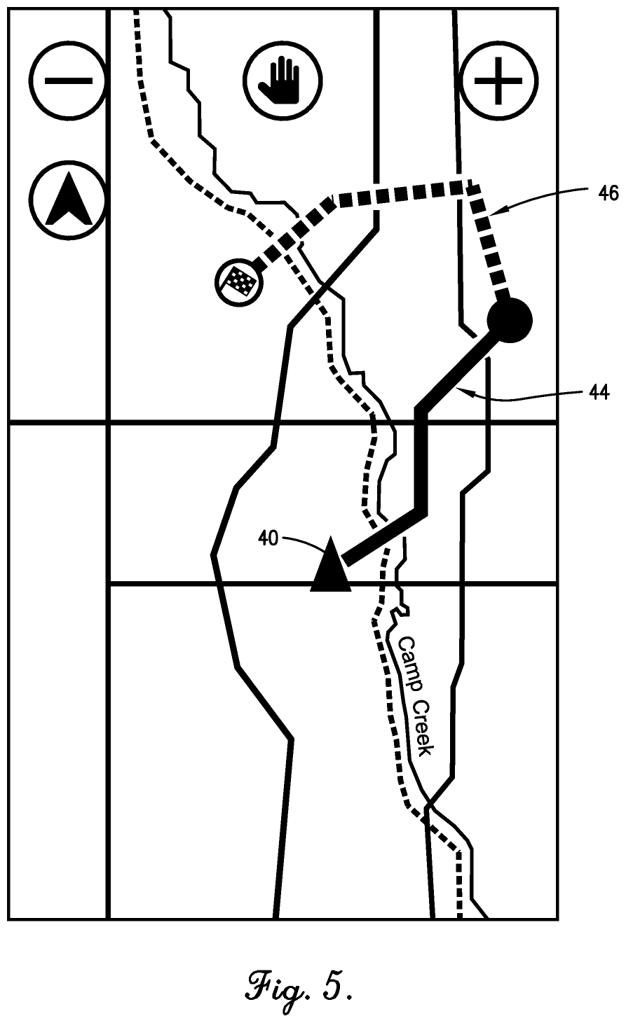

As the user begins riding the eBike 10 over the selected route, the user's real-time position (as determined by the location-determining elements 24) may be continuously updated and presented, via the user icon, at the appropriate location on the graphical map via the user icon. Furthermore, the device 12 may update the route-charge highlighting of the selected route to revise such highlighting based on real-time readings of the charge level of the eBike's 10 battery as the user traverses the selected route. For example, FIG. 5 illustrates a GUI with an updated map after the user has started traveling along the selected route. As an initial matter, it is noted that the user's position has been updated, via the location of the user icon 40, to indicate that the user has completed the first half of the selected route and is currently positioned at the halfway point of the selected route. The device 12 can obtain the current charge level of the eBike's 10 battery (after completing the first half of the selected route) and update the route-charge highlighting. As shown on the updated map, along the third quarter 44 of the selected route, the eBike's 10 battery is predicted to have a charge of more than fifty percent. As such, third quarter 44 of the selected route is highlighted with a solid line type (indicative of the battery having a sufficient state of charge). Along the fourth quarter 46 of the selected route, the eBike's 10 battery is predicted to have a charge between twenty-five and fifty percent. As such, fourth quarter 46 of the selected route is highlighted with a dashed line type (indicative of the battery having a moderately deficient state of charge). Thus, based on the updated route-charge highlighting, the eBike's 10 battery is not estimated to run out of power during the user's ride over the selected route. Such a change from the initial route-charge highlighting shown in FIG. 4 may be due to various reasons, such as the user not using as much motor assistance from the eBike 10 during the first half of the selected route. Thus, embodiments provide for the user's position (as indicated by the user icon 40) and the route-charge highlighting to be continuously updated, in real-time as the user travels over the selected route with the eBike 10.

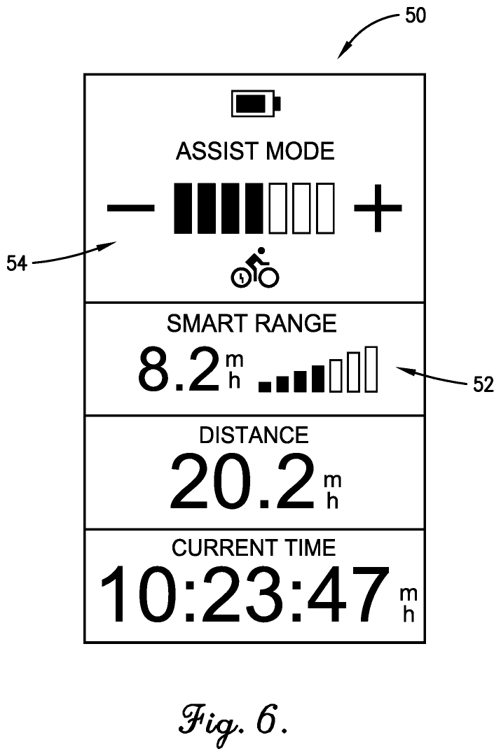

In addition, to presenting a graphical map of the selected route (and surrounding geographical area), the device 12 may be able to provide various other real-time information related to the user's ride over the selected route (i.e., “ride data”). In some embodiments, the ride data may be presented to the user via the graphic display 26 and/or stored, as user data, in the memory element 22 of the device 12. For example, as illustrated by FIG. 6, the graphic display 26 may present ride data in the form of an indication of a real-time charge level of the eBike's 10 battery, such as illustrated by battery icon 50 that is filled to an amount that corresponds with the real-time charge level of the battery (e.g., fully filled battery icon 50 corresponds with a fully charged battery and unfilled battery icon 50 corresponds with a non-charged or dead battery). Such real-time charge level may be updated throughout the user's ride over the selected route. In addition, the graphic display 26 may present ride data in the form of a “smart range” visual element 52, which is an estimated distance over which the eBike may provide motor assistance based on the current charge level of the eBike's 10 battery and/or the anticipated amount of motor assistance. In addition, however, the graphic display may illustrate the current amount of motor assistance being provided by the eBike 10, in the form of motor assistance visual element 54. Embodiments may provide the user with options to permit the user to manually change the amount of motor assistance being provided by the eBike 10. For example, if the user manually increases the amount of motor assistance being provided by the eBike 10 (e.g., by touching the “plus sign” icon on the motor assistance visual element 54 of the GUI shown FIG. 6), the device 12 may immediately update the displayed smart range shown via the smart range visual element 52. Such a motor assistance increase will generally decrease the displayed smart range, as an increase in motor assistance will more quickly deplete the charge level of the eBike's 10 battery. The user may choose to increase the amount of motor assistance if the user is becoming physically tired or needs to complete the selected course in a shorter amount of time. In contrast, if the user manually decreases the amount of motor assistance being provided by the eBike 10 (e.g., by touching the “minus sign” icon on the motor assistance visual element 54 of the GUI shown on the graphic display 26), the device 12 may immediately update the displayed smart range. Such a motor assistance decrease will generally increase the displayed smart range, as a decrease in motor assistance will less quickly deplete the charge level of the eBike's 10 battery. The user may choose to decrease the amount of motor assistance if the user wants to exert more physical effort when completing the selected route.

The graphic display 26 of the device 12 may present other real-time ride data to the user as the user travels over the selected route on the eBike 10. Such ride data may include: the amount of motor assistance used as the user has been riding over the selected route, the amount of time the user has been riding over the selected route, the estimated remaining time the user is estimated to ride to complete the selected route, the current date and/or time, the total distance the user has ridden, the estimated remaining distance the user will ride to complete the selected route, the amount of elevation increase the user has experienced, the remaining amount of elevation increase the user will experience to complete the selected route, the amount of elevation decrease the user has experienced, the remaining amount of elevation decrease the user will experience to complete the selected route, the user's current travel speed, the user's pedaling rate, and the user's current travel direction. The ride data presented to the user may include real-time fitness metrics, such as the user's current heart rate (as perhaps obtained by a biometric sensor associated with the device 12), the number of calories burned during the user's ride over the selected route, the user's VO₂ max, and the like. The graphic display 26 may also present real-time weather data, such as may be obtained from a remote server. Such weather data may include temperature, wind speed, wind direction, precipitation information, sunrise time, sunset time, and the like.

Once the user has completed riding the selected route, the device 12 may store ride data, which is associated with the user's ride over the selected route. The user's ride over the selected route may now being considered a “past ride,” with the ride data of such past ride being associated with the past ride in the memory elements 22 of the device 12. Such ride data may include any of the ride data discussed in the previous paragraph and may specifically include: the amount of motor assistance and/or battery usage used during the past ride. Furthermore, the device 12 may associate specific portions of the selected route of the past ride with actual amounts of motor assistance and/or battery usage used during such portions (as will be discussed in more detail below). The ride data for the past ride may additionally include a total distance traveled over the selected route, a total time taken to travel over the selected route, an average speed of the eBike over the selected route, the total ascent/descent of the over the selected route, and/or the grades of hills climbed with the over the selected route. The ride data may also include health metrics of the user associated with the user's ride over the selected route, such as the user's average heart rate, the number of calories burned, the user's VO₂ max, and the like. All of such ride data may be stored on the memory elements 22 of the device 12 and may be considered part of the user data. In some additional embodiments, the ride data may be transmitted, via the communications element 30, to a remote server for storage and/or for further analysis.

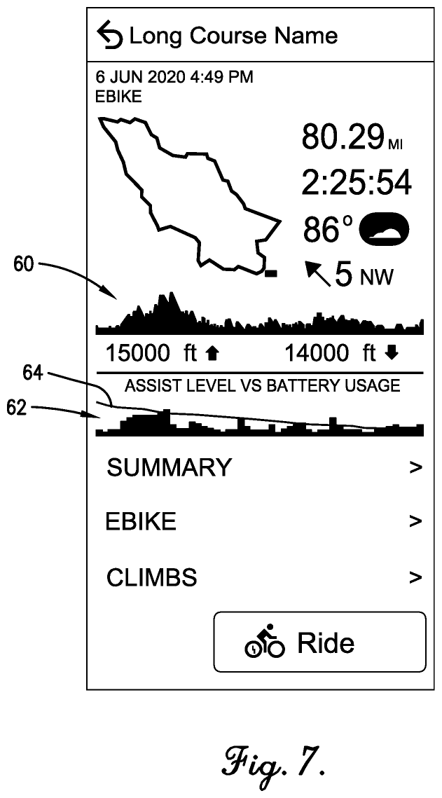

In some embodiments, the device 12 may present portions of the ride data from past rides via the graphic display 26. Such past rides may include routes the user has previously completed with eBike 10. For example, as shown on FIG. 7, ride data for a past ride is illustrated to include a graphical image of a map of the route (e.g., the closed-circuit route) the user previously completed with the eBike 10. The map of the route may be highlighted to indicate locations where motor assistance was or was not used, recommendations where the user should or should not have used motor assistance based on desired range and/or fitness level, combinations thereof, and the like. Various other ride data related to the past ride is shown beside the map of the route, including the total distance of the route, the time taken to complete the route, and weather information for the day the route was completed.

In addition, terrain data, such as elevation information for the route can be provided, such as an elevation chart 60, which shows an elevation cross section of elevation values from the start of the route to the end of the route. In addition, below the elevation chart 60, ride data in the form of a motor assist chart 62 may be presented that shows the actual amount of motor assistance used during the user's ride over the route. As such, the user can visually associate how much motor assistance from the eBike's 10 motor was provided during each portion of the route. As shown, the motor assist chart 62 may be aligned with the elevation chart 60. As such, the device 12 is configured to compare actual amounts of motor assistance used during the user's ride over individual portions of the route with elevation changes over those individual portions of the route, so that the user can determine how elevation changes experienced over the route affect the user's need for motor assistance from the eBike 10. In the exemplary ride data shown in FIG. 7, it can be seen that the highest levels of motor assist were provided as the user was riding the eBike 10 over the portions of the route with largest elevation gains. In addition, the charge level of the battery of the eBike 10 can be plotted over the route, as illustrated by the battery usage plot 64. In some embodiments, the battery usage plot 64 will be plotted over the motor assist chart 62 so as to provide the user a comparison of battery usage and motor assist over the route.

It should be understood that the device 12 may be configured to present other ride data as well, such as level of motor assistance versus: speed of the eBike 10, fitness metrics (e.g., heart rate), terrain data (e.g., elevation changes, ascent/decent, grade of climbs etc.). Similarly, the device 12 may be configured to present the eBike's battery level and/or usage versus: speed of the eBike 10, fitness metrics (e.g., heart rate), terrain data (e.g., elevation changes, ascent/decent, grade of climbs, etc.). Thus, embodiments allow the user to easily compare motor assistance of the eBike to terrain and performance. Such information may be useful to the user in selecting motor assistance in the future in a manner that meets the user's needs, e.g., changing when and how motor assistance is engaged to maximize range and/or maximize (or minimize) physical effort.

The user may use the ride data for the past ride (and/or for multiple past rides) to improve the user's performance on future rides. The user may review the ride data, including motor assistance and/or battery usage as illustrated in FIG. 7, to identify locations on the past ride where the user may reduce and/or increase motor assistance. For example, if the user identifies a portion of the route over which the user used too much motor assistance, the user can be aware of such overuse for the next time the user rides over the route with the eBike. As such, when the user rides over the particular portion of the route the next time, the user can reduce the amount of motor assistance used. Such a reduction in motor assistance may be helpful to increase the range of the eBike 10, particularly in instances where the eBike's 10 battery did not have sufficient charge to complete past rides over the particular route. Alternatively, the user may use the ride data to determine portions of the route where the user became physically tired, so as to provide an indication of where an increased amount of motor assistance would be helpful. Thus, the device 12 is configured to compare the actual amount of motor assistance used during the user's ride over a particular route with amounts of motor assistance used during the user's previous rides over the particular route, such that the user can receive an indication as to whether a ride performance of the user over the particular route has improved.

As has been noted previously, all of such ride data may be stored in the memory elements 22 of the device 12, such that the user can analyze such ride data at later times (e.g., so that the user can compare the user's past performances). For example, the user may analyze how much motor assistance and/or battery charge the user required from the eBike 10 over a series of two or more past rides. As such, the user can determine if the user's performance has improved, such as by the ride data showing a (i) decrease in the amount of motor assistance and/or battery charge needed to complete a particular route during more recent rides, or (ii) a decrease in the amount of time taken to complete a particular route for more recent rides. The user may also use the ride data from past rides to determine if the user's fitness metrics have improved, such as heart rate, VO₂ max, and/or any other health-related data that is collected or monitored by the device 12 improving during more recent rides.

In addition, ride data associated with the user's past rides may be stored in the memory elements 22 of the device 12 as user data, such that the ride data may be used by the device 12 to determine anticipated amounts of motor assistance that should be supplied by the eBike 10 during the user's riding of a particular route. Such ride data may comprise data associated with the user's past rides along the particular route. Alternatively, or in addition, such ride data may comprise data associated with the user's past rides along one or more other routes that have terrain similar to the particular route (e.g., similar elevation changes, similar number, categories, and/or grades of climbs, similar total distances, etc.). In addition, ride data associated with other user's past rides may be used by the device 12 to determine the anticipated amount of motor assistance that should be supplied by the eBike during the user's riding of a particular route. Such ride data for other user's past rides may be stored on the device 12 or may be stored on a remote server and accessed by the device 12. The ride data of other users may comprise data associated with the other users' past rides along the particular route. Alternatively, or in addition, such ride data may comprise data associated with the other users' past rides along one or more other routes that have terrain similar to the particular route (e.g., similar elevation changes, similar number, categories, or grades of climbs, similar total distances, etc.). Thus, the memory elements 22 of the device 12 may include a database of the user's, or other users', ride data for past rides (e.g., actual amounts of past motor assistance) that can be used to estimate the actual amount of motor assistance the user is likely to demand for future rides. Such ride data from one or more users may be aggregated in the database, such that the device 12 can generate a model of anticipated motor assistance. Such a model may be particularly useful in instances where the ride data from past rides is used to estimate an anticipated amount motor assistance that will be required for the user riding over a new route with terrain that is similar to terrain associated with routes from past rides (e.g., similar elevation changes, similar number, categories, or grades of climbs, similar total distances, etc.).

Although the above functions and features of embodiments of the present invention have been described as being performed, at least in part, by the portable electronic device 12, it should understood that in some embodiments, certain of such functions and features may be performed by a remote computing system or server (e.g., cloud-based). As such, the device 12 may access such a remote system that performs the various functions and features described herein.

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

1. A portable electronic device configured to be used by a user riding an electric bicycle (eBike), wherein the eBike comprises a battery and an electric motor, wherein the electric motor is configured to selectively provide motor assistance to the user in propelling the eBike, said portable electronic device comprising:
one or more memory elements, wherein the memory elements are configured to store map data and user data, wherein the map data comprises distance information and terrain information for a plurality of routes, and wherein the user data comprises information indicative of a fitness level of the user; and
a display screen configured to present information to the user,
wherein said portable electronic device is configured to—
connect in data communication with the eBike,
receive an indication of a selected route over which the user intends to ride the eBike,
determine, from the map data and the user data, an anticipated amount of motor assistance to be provided by the electric motor during the user's ride over the selected route,
obtain an indication, via the data communication with the eBike, of a charge level of the battery of the eBike,
determine, based on the anticipated amount of motor assistance and the charge level of the battery, whether the charge level of the battery of the eBike is sufficient to complete the user's ride over the selected route, and
present, via the display screen, an indication of whether the charge level of the battery of the eBike is sufficient to complete the user's ride over the selected route.

2. The portable electronic device of claim 1, wherein said portable electronic device includes an attachment assembly configured to releasably attach said portable electronic device to the eBike.

3. The portable electronic device of claim 1, wherein said portable electronic device is selected from an eBike computer, a smartphone, and a wearable device.

4. The portable electronic device of claim 1, wherein said portable electronic device is configured to connect in data communication with the eBike wirelessly.

5. The portable electronic device of claim 1, wherein the terrain information comprises information associated with elevation changes over the selected route.

6. The portable electronic device of claim 1, wherein the user data includes one or more of the following: an age of the user, a sex of the user, an average heart rate of the user, a body mass index of the user, and a VO₂ max of the user.

7. The portable electronic device of claim 1, wherein the user data comprises past performance data related to the user previously riding the eBike over the selected route or related to the user previously riding the eBike over one or more other routes with similar terrain as the selected route.

8. The portable electronic device of claim 1, wherein upon the portable electronic device determining that the charge level of the battery of the eBike is insufficient to complete the user's ride over the selected route, the portable electronic device is configured to generate an alert.

9. The portable electronic device of claim 8, wherein upon the portable electronic device determining that the charge level of the battery of the eBike is insufficient to complete the user's ride over the selected route, the portable electronic device is configured to receive from the user an adjustment of the anticipated amount of motor assistance, and wherein the portable electronic device is further configured to determine, based on the adjusted anticipated amount of motor assistance and the charge level of the battery, whether the charge level of the battery of the eBike is sufficient to complete the user's ride over the selected route.

10. The portable electronic device of claim 8, wherein upon the portable electronic device determining that the charge level of the battery of the eBike is insufficient to complete the user's ride over the selected route, the portable electronic device is further configured to present one or more options of other routes for which the battery of the eBike has a sufficient charge level to complete a ride of the user.

11. The portable electronic device of claim 1, wherein upon the portable electronic device determining that the charge level of the battery of the eBike is sufficient to complete the user's ride over the selected route, the portable electronic device is configured to present a graphical map of the selected route and a geographical area around the selected route.

12. The portable electronic device of claim 11, wherein the selected route presented on the graphical map is segmented, and wherein each segment of the selected route is highlighted to illustrate an estimated battery level of the battery of the eBike when the user travels over the relevant segment of the selected route.

13. The portable electronic device of claim 12, wherein upon the user starting to ride the eBike over the selected route, the portable electronic device is configured to present on the graphical map an icon indicative of a real-time location of the portable electronic device, and wherein the estimated battery level of the battery over each segment is updated in real-time.

14. The portable electronic device of claim 1, wherein upon the user completing the selected route, said portable electronic device is configured to store ride data related to the user's ride over the selected route.

15. The portable electronic device of claim 14, wherein the ride data includes an actual amount of motor assistance used during the user's ride over the selected route.

16. The portable electronic device of claim 15, wherein the portable electronic device is configured to compare the actual amounts of motor assistance used during the user's ride over individual portions of the selected route with elevation changes over those individual portions of the selected route, such that the user can determine how elevation changes of over the selected route affect a need of the user for motor assistance from the eBike.

17. The portable electronic device of claim 15, wherein said portable electronic device is configured to compare the actual amount of motor assistance used during the user's ride over the selected route with amounts of motor assistance used during the user's previous rides over the selected route, such that the user can receive an indication as to whether a ride performance of the user over the selected route has improved.

18. A portable electronic device configured to be used by a user riding an electric bicycle (eBike), wherein the eBike comprises a battery and an electric motor, wherein the electric motor is configured to selectively provide motor assistance to the user in propelling the eBike, said portable electronic device comprising:
one or more memory elements, wherein the memory elements are configured to store map data and user data, wherein the map data comprises distance information and terrain information for a plurality of routes, wherein the terrain information comprises information associated with elevation changes for the plurality of routes; and
a display screen configured to present information to the user,
wherein said portable electronic device is configured to—connect in data communication with the eBike,
receive an indication of a selected route over which the user intends to ride the eBike,
determine, from the map data and the user data, an anticipated amount of motor assistance to be provided by the electric motor during the user's ride over the selected route, wherein the user data comprises past performance data related to the user previously riding the eBike over the selected route or to the user previously riding the eBike over one or more other routes with similar terrain as the selected route,
obtain an indication, via the data communication with the eBike, of a charge level of the battery of the eBike,
determine, based on the anticipated amount of motor assistance and the charge level of the battery, whether the charge level of the battery of the eBike is sufficient to complete the user's ride over the selected route, and
present, via the display screen, an indication of whether the charge level of the battery of the eBike is sufficient to complete the user's ride over the selected route.

19. The portable electronic device of claim 18, wherein the one or more other routes have similar terrain as the selected route if the one or more other routes have similar elevation changes as the selected route.

20. The portable electronic device of claim 18, wherein the one or more other routes have similar terrain as the selected route if the one or more other routes have a similar total distance as the selected route.
